(19) United States
(12) Patent Application Publication
(10) Pub. No.: US 2024/0142455 A1
(43) Pub. Date: May 2, 2024

(54) METHOD OF DIAGNOSING EARLY-STAGE NON-SMALL CELL LUNG CANCER

(71) Applicant: BIOMARK CANCER SYSTEMS INC., Richmond, B.C. (CA)

(72) Inventors: Rashid Ahmed M. Bux, Richmond, B.C. (CA); David Wishart, Richmond, B.C. (CA)

(21) Appl. No.: 17/769,353

(22) PCT Filed: Oct. 17, 2020
(86) PCT No.: PCT/CA2020/051398

§ 371 Date: Apr. 15, 2022

Related U.S. Application Data
(60) Provisional application No. 62/916,486, filed on Oct. 17, 2019.

(51) Int. Cl. G01N 33/574

(52) U.S. Cl. CPC G01N 33/57423; G01N 33/57488

(57) ABSTRACT

The present disclosure pertains to a method of diagnosing cancer and, in particular, to a method of diagnosing early-stage non-small cell lung cancer by measuring metabolite biomarkers in serum and plasma. In some aspects, the methods comprise determining the concentration metabolites from the group comprising β-hydroxybutyric acid, LysoPC 20:3, PC ae C40:6, citric acid, carnitine, Sand fumaric acid. In some aspects, the methods comprise determining the concentration metabolites from the group comprising β-hydroxybutyric acid, LysoPC 20:3, spermidine, and fumaric acid.

### RELATED APPLICATIONS

This application claims priority to U.S. patent application No. 62/916,486, the contents of which are incorporated herein by reference.

### TECHNICAL FIELD

The present disclosure relates to a method of diagnosing cancer and, in particular, to a method of diagnosing early-stage non-small cell lung cancer by measuring metabolite biomarkers in serum and plasma.

### BACKGROUND

Lung cancer is the leading cause of cancer-related deaths worldwide. Sensitive, accurate strategies for the early detection of lung cancer are essential for improving lung cancer survival statistics. Unfortunately, current methods for the detection or screening of lung cancer are not ideal. Although low dose computed tomography (LDCT) scan has been shown to reduce lung cancer mortality, broad clinical implementation is hampered by several technical and socio-economical challenges. Therefore, the development of a low-cost, minimally invasive assay for early-stage lung cancer detection would significantly improve the current situation.

International Patent Application Publication No. WO 2016/205960 which was published on Dec. 29, 2016, discloses a biomarker panel for a serum test for detecting lung cancer detects a biomarker selected from the group of biomarkers consisting of valine, arginine, ornithine, methionine, spermidine, spermine, diacetylspermine, C10:2, PC aa C32:2, PC ae C36:0, and PC ae C44:5; and lysoPC a C18:2, or a combination thereof.

### SUMMARY OF THE DISCLOSURE

Aspects of this disclosure relate to a method, the method comprising determining the concentration of each metabolite of a group of metabolites in a biological sample from a subject, wherein the group of metabolites comprises: β-hydroxybutyric acid, LysoPC 20:3, PC ae C40:6, citric acid, carnitine, and fumaric acid; β-hydroxybutyric acid, LysoPC 20:3, PC ae C40:6, and fumaric acid; or β-hydroxybutyric acid, PC ae C40:6, citric acid, and carnitine. In various embodiments, the disclosed method is a method of diagnosing non-small cell lung cancer and, in particular embodiments, stage I or stage II non-small cell lung cancer.

Aspects of this disclosure relate to a method, the method comprising determining the concentration of each metabolite of a group of metabolites in a biological sample from a subject, wherein the group of metabolites comprises β-hydroxybutyric acid, LysoPC 20:3, fumaric acid, and spermine. In various embodiments, the disclosed method is a method of diagnosing non-small cell lung cancer and, in particular embodiments, stage I non-small cell lung cancer.

Aspects of the disclosure relate to treatment of patients for non-small cell lung cancer once diagnosed according to a method as described herein.

### BRIEF DESCRIPTION OF THE DRAWINGS

FIG. 1a is a 2-D partial least squares discriminant analysis (PLS-DA) plot showing the comparison between plasma metabolite data acquired for healthy controls (shown in shaded area on left) vs. stage I NSCLC patients (shown in shaded area on right);

FIG. 1b is a variable importance in projection (VIP) plot showing the most discriminating metabolites for healthy controls vs. stage I NSCLC patients. The boxes indicate whether metabolite concentration is increased (circled) or decreased (not circled) in controls vs. cases.;

FIG. 2a is a 2-D partial least squares discriminant analysis (PLS-DA) plot showing the comparison between plasma metabolite data acquired for healthy controls (shown in shaded area on left) vs. all stages NSCLC patients (shown in shaded area on right). PLS-DA results of healthy controls vs. all stages NSCLC;

FIG. 2b is a variable importance in projection (VIP) plot showing the most discriminating metabolites for healthy controls vs. all stages NSCLC patients. The boxes indicate whether metabolite concentration is increased (circled) or decreased (not circled) in controls vs. cases.;

FIG. 3a is a receiver operating characteristic (ROC) curve generated by the metabolite-only logistic regression model for diagnosing stage I NSCLC patients. ROC curves and their 95% CI on the discovery set is shown with curved line. ROC curves obtained from the validation set is shown with line resembling step function;

FIG. 3b is a receiver operating characteristic (ROC) curve generated by the metabolites+smoking history logistic regression model for diagnosing stage I NSCLC patients. ROC curves and their 95% CI on the discovery set is shown with curved line. ROC curves obtained from the validation set is shown with line resembling step function;

FIG. 4a is a receiver operating characteristic (ROC) curve generated by the random forest exploration models for stage I NSCLC patients with different numbers of metabolite features. Number of metabolite features in each model are indicated as Var. in the left-bottom box;

FIG. 4b is a variable importance in projection (VIP) plot showing the most frequently selected metabolites for healthy controls vs. stage I NSCLC patients (Number of features=5). The boxes indicate whether metabolite concentration is increased (circled) or decreased (not circled) in controls vs. cases;

FIG. 5a is a 2-D partial least squares discriminant analysis (PLS-DA) plot showing the comparison between plasma metabolite data acquired for healthy controls (shown in shaded area on left) vs. stage II NSCLC patients (shown in shaded area on right);

FIG. 5b is a variable importance in projection (VIP) plot showing the most discriminating metabolites for healthy controls vs. stage II NSCLC patients. The boxes indicate whether metabolite concentration is increased (circled) or decreased (not circled) in controls vs. cases;

FIG. 6a is a receiver operating characteristic (ROC) curve generated by the random forest exploration models for stage II INSCLC patients;

FIG. 6b is a variable importance in projection (VIP) plot showing the most frequently selected metabolites for stage II INSCLC patients. (Number of features=5). The boxes indicate whether metabolite concentration is increased (circled) or decreased (not circled) in controls vs. cases;

FIG. 7a is a receiver operating characteristic (ROC) curve generated by the metabolite-only logistic regression model for diagnosing stage II NSCLC patients. Number of metabolite features in each model are indicated as Var. in the left-bottom box. ROC curves and their 95% CI on the discovery set is shown with curved line. ROC curves obtained from the validation set is shown with line resembling step function;

FIG. 7b is a receiver operating characteristic (ROC) curve generated by the metabolites+smoking history logistic regression model for diagnosing stage II NSCLC patients. ROC curves and their 95% CI on the discovery set is shown with curved line. ROC curves obtained from the validation set is shown with line resembling step function;

FIG. 8a is a 2-D principal component analysis (PCA) scores plot showing the comparison between plasma metabolite data acquired for healthy controls (shown in shaded area on bottom) vs. all stages NSCLC patients (shown in shaded area on top);

FIG. 8b is a partial least squares discriminant analysis (PLS-DA) plot showing the comparison between plasma metabolite data acquired for healthy controls (shown in shaded area on left) vs. all stages NSCLC patients (shown in shaded area on right);

FIG. 8c is a variable importance in projection (VIP) plot showing the comparison between plasma metabolite data acquired for healthy controls vs. all stages NSCLC patients. The most discriminating metabolites are shown in descending order of coefficient scores. The boxes indicate whether metabolite concentration is increased (circled) or decreased (not circled) in controls vs. cases;

FIG. 9a is a receiver operating characteristic (ROC) curve generated by the metabolite-only logistic regression model for diagnosing early stages (stage I+II) NSCLC patients. ROC curves and their 95% CI on the discovery set is shown with curved line. ROC curves obtained from the validation set is shown with line resembling step function;

FIG. 9b is a receiver operating characteristic (ROC) curve generated by the metabolites+smoking history logistic regression model for diagnosing early stages (stage I+II) NSCLC patients. ROC curves and their 95% CI on the discovery set is shown with curved line. ROC curves obtained from the validation set is shown with line resembling step function.

FIG. 10 is a partial least squares discriminant (PLS-DA) analysis plot showing 2D-scores plot of quantitative MS metabolite analysis of serum samples from stage I lung cancer patients compared to healthy controls;

FIG. 11 is a variable importance in projection (VIP) analysis plot ranking discriminating serum metabolites in descending order of importance. This plot comes from PLS-DA and ranks the metabolites in order of importance for classifying stage I cancer. A variable importance plot (VIP) score (x-axis) higher than a coefficient of 85 indicates highly significant metabolites. The right panel shows whether a specific metabolite is increased or decreased in lung cancer relative to healthy controls. So LysoPC-20:3 is increased in lung cancer while spermine is deceased in lung cancer;

FIG. 12 is a receiver operating characteristic (ROC) analysis of lung cancer metabolites in serum from stage I lung cancer patients, including the four most important metabolites from VIP analysis of serum samples shown in FIG. 11; and

FIG. 13 is a receiver operating characteristic (ROC) analysis of lung cancer metabolites (the four most important metabolites from VIP analysis of in serum serum samples shown in FIG. 11) from stage I lung cancer patients with smoking status included in the model. The permutation test (with 1000 repetitions) for the ROC analysis indicates this result is significant with a p-value <0.001.

### DEFINITIONS

“Smoker” as used herein includes a “current smoker” and a “former smoker” as defined in the “Tobacco Glossary” of National Center for Health Statistics (“NCHS”) of the Centers for Disease Control and Prevention (“CDC”).

“Non-smoker” as used herein is a subject that is not a “Smoker” as defined above, including a “Never smoker”.

“Amount of Smoking” as used herein is a value calculated by multiplying the period of smoking (in days) by the daily amount of smoking (cig/day).

### DETAILED DESCRIPTION

There is disclosed a set of high-performing (AUC>0.9) plasma metabolite biomarkers for detecting early-stage non-small cell lung cancer (NSCLC). Plasma samples were acquired from 156 patients with biopsy-confirmed NSCLC along with age and gender-matched plasma samples from 60 healthy controls. Clinical data and smoking history were also available for all samples. A fully quantitative targeted mass spectrometry (MS) analysis (direct injection/LC and tandem MS) was performed on all 216 plasma samples. Two thirds of the samples were randomly selected and used for discovery and one third for validation. Metabolite concentration data, clinical data and smoking history were used to determine optimal sets of biomarkers and optimal regression models for identifying different stages of NSCLC using the discovery sets. The same biomarkers and regression models were used and assessed on the validation models.

An average of 103 metabolites were quantified in these plasma samples. Univariate and multivariate statistical analysis identified β-hydroxybutyric acid, LysoPC 20:3, PC ae C40:6, citric acid and fumaric acid as being significantly different between healthy controls and stage I/II NSCLC. Robust predictive models with areas under the curve (AUC)>0.9 were developed and validated using these metabolites and other, easily measured clinical data for detecting different stages of NSCLC.

Archived plasma samples were obtained from the IUCPQ (Institut Universitaire de Cardiologie et de Pneumologie de Quebec) Tissue Bank, which is the site of the Respiratory Health Network Tissue Bank of the Fonds de la Recherché du Quebec-Sante in Quebec, Canada. Frozen (−80° C.) aliquots of 200-400 μL of plasma were assembled and shipped to The Metabolomic Innovation Centre (TMIC) at the University of Alberta, Canada for quantitative metabolomic analysis. The plasma samples were collected from 156 patients with biopsy-proven and biopsy-graded NSCLC and 60 healthy controls with comparable age and gender profiles. Healthy controls consisted of both smokers and non-smokers. The cancer samples had detailed data on cancer stage, lung cancer histology, age, weight, height, body mass index, smoking status (never/former/current), smoking history (cig/day and period of smoking in years), sex, survival history, medical condition history, personal history of cancer, lung disease status, treatment, tumor size (in mm), tumor grading, details of positive nodules, as well as data collected on each cancer patient's transthoracic needle biopsy, transbronchial biopsy, endobronchial biopsy, bronchoalveolar lavage, bronchial brushing, bronchial aspiration, endobronchial ultrasound, transesophageal echocardiography, bone scintigraphy, abdominal ultrasound, abdominal CT scan, thoracic CT scan, cerebral CT scan, thoracic X-ray, mediastinoscopy, thoracic MRI, cerebral MRI, and PET scan. Healthy controls had data on age, weight, height, body mass index, smoking status (never/former/current), smoking history (cig/day and period of smoking in years), and medical condition history. Patients (and controls) with a history of any liver or kidney disease, and any previous treatment with anti-neoplastic drugs were excluded from this cohort.

Optima™ LC/MS grade formic acid and HPLC grade water were purchased from Fisher Scientific (Ottawa, ON, CA). Sixty-eight pure reference standard compounds were purchased from Sigma-Aldrich (Oakville, ON, CA). Optima™ LC/MS grade Ammonium acetate, phenylisothiocyanate (PITC), 3-nitrophenylhydrazine (3-NPH), 1-ethyl-3-(3-dimethylaminopropyl) carbodiimide (EDC) and butylated hydroxytoluene (BHT), HPLC grade pyridine, HPLC grade methanol, HPLC grade ethanol and HPLC grade acetonitrile (ACN) were also purchased from Sigma-Aldrich (Oakville, ON, CA). Forty-four ²H-, ¹³C- and ¹⁵N-labelled compounds, which were used as internal quantification standards for amino acids, biogenic amines, carnitines and derivatives, phosphatidylcholines and their derivatives were purchased from Cambridge Isotope Laboratories, Inc. (Tewksbury, MA, USA). 3-(3-hydroxyphenyl)-3-hydroxypropionic acid (HPHPA) and ¹³C-labelled HPHPA were synthesized in-house as described by Khaniani et al., “A Simple and Convenient Synthesis of Unlabeled and 13C-Labeled 3-(3-Hydroxyphenyl)-3-Hydroxypropionic Acid and Its Quantification in Human Urine Samples”, Metabolites, 2018, 8(4):80. All other standards including lactic acid, beta-hydroxybutyric acid, alpha-ketoglutaric acid, citric acid, butyric acid, isobutyric acid, propionic acid, p-hydroxyhippuric acid, succinic acid, fumaric acid, pyruvic acid, hippuric acid, methylmalonic acid, homovanillic acid, indole-3-acetic acid, uric acid and their isotope-labelled standards were all purchased from Sigma-Aldrich (Oakville, ON, CA). Multiscreen “solvinert” filter plates (hydrophobic, PTFE, 0.45 m, clear, non-sterile) and Nunc® 96 DeepWell™ plates were purchased from Sigma-Aldrich (Oakville, ON, CA).

All solid chemicals were carefully weighed on a CPA225D semi-micro electronic balance (Sartorius, USA) with a precision of 0.0001 g. Stock solutions of each compound were prepared by dissolving the accurately weighed solids in water. Calibration curve standards were obtained by mixing and diluting the corresponding stock solutions with water. For amino acids, biogenic amines, carbohydrates, carnitines and derivatives, phosphatidylcholines and their derivatives, stock solutions of isotope-labelled compounds were also prepared in the same way. A working internal standard (ISTD) solution mixture in water was also made by mixing all the prepared isotope-labeled stock solutions together. For organic acids, stock solutions of isotope-labelled compounds were prepared by dissolving the accurately weighed solids in 75% aqueous methanol. A working internal standard (ISTD) solution mixture in 75% aqueous methanol was made by mixing and diluting all the isotope-labelled stock solutions. All standard solutions were aliquoted and stored at −80° C. until further use.

A targeted, quantitative MS-based metabolomics approach was used to analyze the plasma samples using a combination of direct injection (DI) mass spectrometry (MS) and reverse-phase high performance liquid chromatography (HPLC) tandem mass spectrometry (MS/MS). This 96-well plate, semi-automated assay, in combination with an ABI 4000 Q-Trap (Applied Biosystems/MDS Sciex) mass spectrometer, can be used for the targeted identification and quantification of up to 138 different endogenous metabolites including amino acids, organic acids, biogenic amines, acylcarnitines, glycerophospholipids, sphingolipids and sugars. The method combines the derivatization and extraction of the 138 analytes, and the selective mass-spectrometric detection using multiple reaction monitoring (MRM) pairs. Isotope-labeled internal standards and other internal standards are integrated into special filter inserts placed inside a 96-well plate for precise metabolite quantification. The assay uses an upper 96 deep-well plate with a 96-well filter plate attached below using sealing tape. The first 14 wells in the upper plate are used for quality control and calibration. The first well serves as a double blank, three wells contain blank samples, seven wells contain reference compound standards and three wells contain quality control samples.

Briefly, plasma samples were thawed on ice (in the dark) and were vortexed and centrifuged at 18,000 rcf (relative centrifugal force or×g). 10 μL of each sample was loaded onto the center of the filter insert on the upper 96-well kit plate and dried in a stream of nitrogen. Subsequently, PITC was added to each well (in the plate) for amine derivatization. After incubation, the filter inserts were dried using an evaporator. Extraction of the metabolites was then achieved by adding 300 μL of methanol containing 5 mM ammonium acetate. The extracts were obtained by centrifugation (at 50 rcf for 5 minutes) of the double plate system. This allowed the contents of the upper 96-well plate to flow into the lower 96-deep well plate. For analysis of biogenic amines and amino acids, extracts were then diluted by water. For analysis of sugars, carnitines and lipids, extracts were diluted with methanol. Mass spectrometric analysis of the diluted extracts was performed on an HPLC (Agilent 1100 HPLC, Agilent Technologies, Santa Clara, US) equipped Qtrap® 4000 tandem mass spectrometry instrument (Applied Biosystems/MDS Analytical Technologies, Foster City, CA).

For the analysis of organic acids, 50 μL of the plasma samples were mixed thoroughly with the ISTD mixture solution and ice-cold methanol and then left in a −20° C. freezer overnight for protein precipitation. After removing the samples from the freezer, all the tubes were centrifuged at 18,000 rpm for 20 min (to spin down the protein precipitate). The supernatant was then transferred to each well of the 96 well plate system, followed by the addition of 25 μL each of the following three reagents: 3-NPH (250 mM in methanol), EDC (150 mM in methanol) and pyridine for a 2-hour derivatization reaction. After the derivatization reaction was complete, water and a BHT solution (2 mg/mL in methanol) were added to dilute and stabilize the final solution. 10 μL was injected into an HPLC-equipped Qtrap® 4000 mass spectrometer for LC-MS/MS analysis.

Recommended statistical procedures for standard quantitative metabolomic analysis were followed. In particular, metabolites with more than 50% of missing values (in all groups) were removed from further analysis. For metabolites with the fraction of missing values below 50%, values were imputed by using half of the minimum concentration value for that metabolite. Median normalization, log transformation and auto-scaling (mean-centered and divided by the standard deviation of each variable) were applied for data scaling and normalization. Feature normality was checked by the Shapiro-Wilk test with a p-value threshold set at 0.05. Univariate analysis of the continuous data and the categorical data were performed by a Student t-test and a Fisher's exact test, respectively. Principal component analysis (PCA) and partial least squares discriminant analysis (PLS-DA) were performed by using MetaboAnalyst. A 1000-fold permutation test was performed to minimize the possibility that the observed separation of the PLS-DA was due to chance.

Logistic regression with a Lasso feature selection algorithm was used to develop predictive models of NSCLC staging using both metabolite and clinical variables. For these regression studies, two thirds of the samples (40 controls, 40-94 cancer samples, depending on staging) were randomly chosen to serve as the discovery sets. 10-fold cross validation was performed on all discovery/training set models. Once the optimal regression models for each cancer stage predictor had been identified the remaining one third of the samples (20 controls and 20-62 cancer samples, serving as a holdout set) were used to validate each of the corresponding regression models. The area under the receiver-operator characteristic curves (AUC), sensitivities/specificities and the 95% confidence intervals were calculated for all of the discovery and the validation sets and all of the models using MetaboAnalyst.

A total of 138 different metabolites were tested by our quantitative LC-MS method. Due to their low abundance, 35 metabolites were removed for having a high (>50%) fraction of missing values. Most of these missing values arose from the fact that the metabolite concentrations in plasma fell below the limit of detection (LOD). Sample numbers in each group are summarized in Table 1 below.

TABLE 1
Summary of grouping of samples

| | Number of | | Gender | | Smoking Status | | |
|---|---|---|---|---|---|---|---|
| Group | Samples | Age | Male | Female | Never | Former | Current |
| Stage I NSCLC | 70 | 49-79 | 26 | 44 | 14 | 40 | 16 |
| Stage II NSCLC | 60 | 49-79 | 20 | 40 | 5 | 51 | 4 |
| Stage IIIB/IV NSCLC | 26 | 42-79 | 14 | 12 | 0 | 18 | 8 |
| Healthy control | 60 | 49-77 | 26 | 34 | 38 | 22 | 0 |
| Total | 216 | 42-79 | 86 | 130 | 57 | 131 | 28 |

Comparisons between the cancer patients and healthy controls regarding age, gender, height, weight, and smoking history (Yes=former+current, No=never) were conducted using standard Student's t-tests or Fisher's Exact Test to confirm their demographic comparability. The only significantly different variable was smoking history (p-value=2.673×10⁻¹³). The effect on lung cancer incidence based on multiple clinical variables, including age, gender, height, weight, and smoking history (Yes=former+current, No=never) were further evaluated by logistic regression. The results are summarized in Table 2 below. As might be expected only smoking history was identified as the clinical variable significantly related to lung cancer incidence (p-value=1.13×10⁻¹¹). Although the correlation between smoking history and lung cancer has been heavily studied and widely accepted, the model suggested it would be a good strategy to integrate smoking history (including duration and amount of smoking) into any diagnostic model for identifying early lung cancer.

TABLE 2
Logistic regression based correlation
study: NSCLC vs. clinical variants

| | Estimate | std. error | z value | p.value |
|---|---|---|---|---|
| (Intercept) | 5.9317 | 5.6113 | 1.0571 | 0.2905 |
| Age | 0.0170 | 0.0232 | 0.7306 | 0.4650 |
| Gender | −0.2930 | 0.5870 | −0.4991 | 0.6177 |
| Height | −4.6740 | 3.3846 | −1.3810 | 0.1673 |
| Weight | −0.0014 | 0.0138 | −0.1041 | 0.9171 |
| Smoking (Y/N) | 2.8079 | 0.4136 | 6.7883 | 1.13*10⁻¹¹ |

By applying a simple Student's t-test to the metabolomics data set, large differences between the metabolic profiles of healthy controls and lung cancer patients (all stages) were revealed. Table 3 below lists the 36 metabolites with significant FDR adjusted p-values (q<0.05) identified via the t-test. In the study, phosphatidylcholines such as PC ae C40:6, PC aa C38:0, and PC aa C40:2 were among the most downregulated metabolites in the plasma of NSCLC patients, while lysophosphatidylcholines (LysoPCs) such as LysoPC 20:3 and LysoPC 20:4 were significantly upregulated in cancer patients. Other significantly altered metabolites included β-hydroxybutyric acid (increased in NSCLC), methionine sulfoxide (decreased), tryptophan (decreased), carnitines (CO and C2, both increased), and members of the TCA cycle such as citrate (decreased) and fumaric acid (increased).

TABLE 3
Metabolites with significant differences between normal cases
and NSCLC patients in univariate statistical analysis.

| Name of metabolites | Fold Change | FDR |
|---|---|---|
| PC ae C40:6 | 1.3631 | 1.42*10⁻¹¹ |
| β-hydroxybutyric acid | 0.24679 | 1.67*10⁻¹¹ |
| PC aa C38:0 | 1.3403 | 2.64*10⁻¹¹ |
| PC aa C40:2 | 1.3534 | 9.01*10⁻¹⁰ |
| Tryptophan | 1.4844 | 4.42*10⁻⁰⁹ |
| Methionine-sulfoxide | 1.5127 | 4.10*10⁻⁰⁸ |
| LysoPC 20:3 | 0.75633 | 1.49*10⁻⁰⁷ |
| PC aa 38:6 | 1.3162 | 2.60*10⁻⁰⁷ |
| C2 | 0.70241 | 3.91*10⁻⁰⁷ |
| Canitine (C0) | 0.73085 | 1.70*10⁻⁰⁶ |
| PC aa C40:6 | 1.3 | 1.70*10⁻⁰⁶ |
| Glutamic acid | 0.68151 | 7.28*10⁻⁰⁶ |
| Citric acid | 1.304 | 7.28*10⁻⁰⁶ |
| PC aa 36:6 | 1.3259 | 7.87*10⁻⁰⁶ |
| Fumaric acid | 0.7102 | 1.85*10⁻⁰⁵ |
| Spermine | 1.1987 | 2.02*10⁻⁰⁵ |
| Succinic acid | 1.1925 | 2.09*10⁻⁰⁵ |
| Glucose | 0.81515 | 6.96*10⁻⁰⁵ |
| Indole acetic acid | 1.184 | 1.05*10⁻⁰⁴ |
| Tyrosine | 1.2665 | 1.06*10⁻⁰⁴ |
| Valine | 0.79395 | 7.41*10⁻⁰⁴ |
| LysoPC 18:2 | 1.2344 | 8.68*10⁻⁰⁴ |
| C18 | 1.1752 | 9.28*10⁻⁰⁴ |
| C18:2 | 0.74435 | 9.57*10⁻⁰⁴ |
| Alanine | 1.1912 | 9.79*10⁻⁰⁴ |
| Betaine | 1.3079 | 0.003633 |
| Ornithine | 0.83172 | 0.005016 |
| SM (OH) C14:1 | 1.1604 | 0.005877 |
| Pyruvic acid | 0.82437 | 0.010011 |
| Trimethylamine N-oxide | 1.65 | 0.011831 |
| LysoPC 20:4 | 0.84613 | 0.013234 |
| Phenylalanine | 1.1402 | 0.013234 |
| Arginine | 0.81737 | 0.014319 |
| SM (OH) C24:1 | 1.1486 | 0.028486 |
| LysoPC 16:1 | 0.85293 | 0.02898 |
| Cadaverine | 0.80986 | 0.039765 |

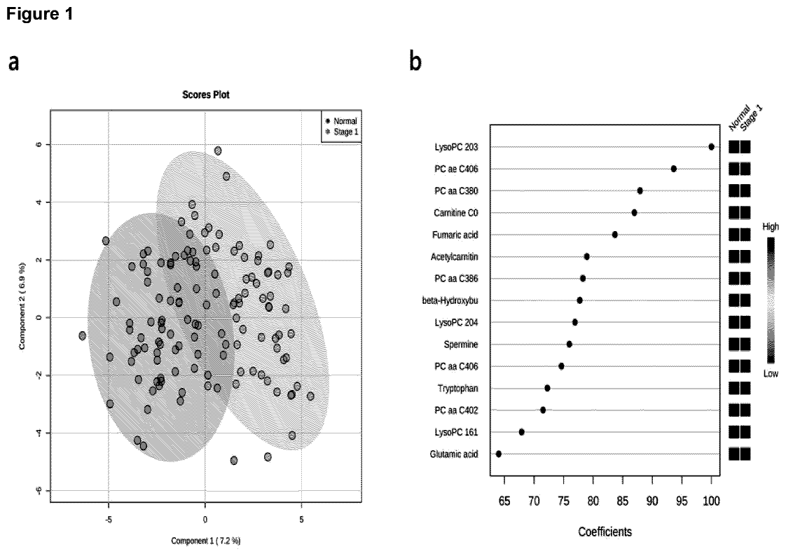

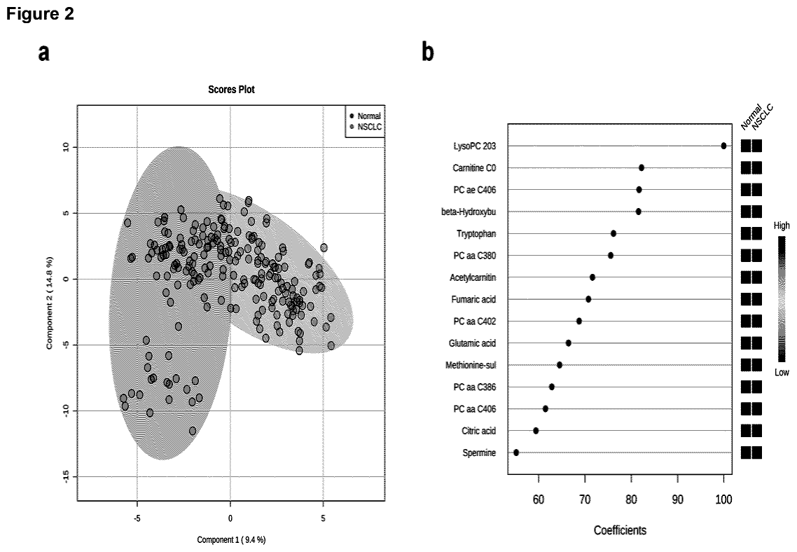

Multivariate analysis was also conducted to further reveal metabolite differences between healthy controls and NSCLC patients at all stages. Using PLS-DA, a clear separation was found between NSCLC patients and healthy controls (FIG. 1a). Permutation testing demonstrated that the observed separation was not by chance (P<0.001). LysoPC 20:3, carnitine, β-hydroxybutyric acid and PC ae C40:6 were found to have the highest overall coefficient score that drove the separation (FIG. 1b).

Biomarkers that can effectively diagnose lung cancer patients in early stages of the disease are obviously more valuable than biomarkers for later stages of the disease. Therefore, a series of statistical analyses was carried out to identify plasma metabolites that could distinguish NSCLC patients at stage I vs. healthy controls. As shown in FIG. 1a, the PLS-DA analysis shows a clearly detectable separation between the stage I NSCLC group and healthy controls. Permutation testing revealed that the observed separation between the cases and controls was not due to chance (p-value<0.001). FIG. 1b displays the results of the overall coefficient scores from the PLS-DA. Based on this analysis, LysoPC 20:3, PC ae C40:6, PC aa C38:0, carnitine and fumaric acid appeared to be the most important plasma metabolites for distinguishing stage I NSCLC patients from healthy controls.

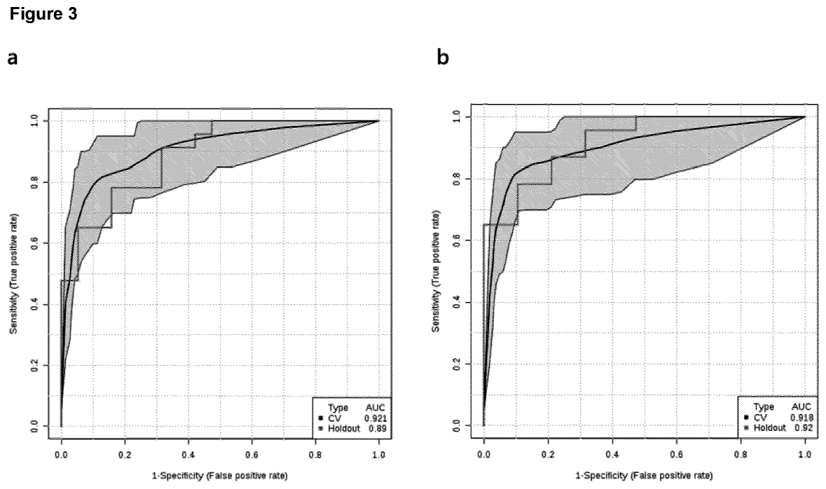

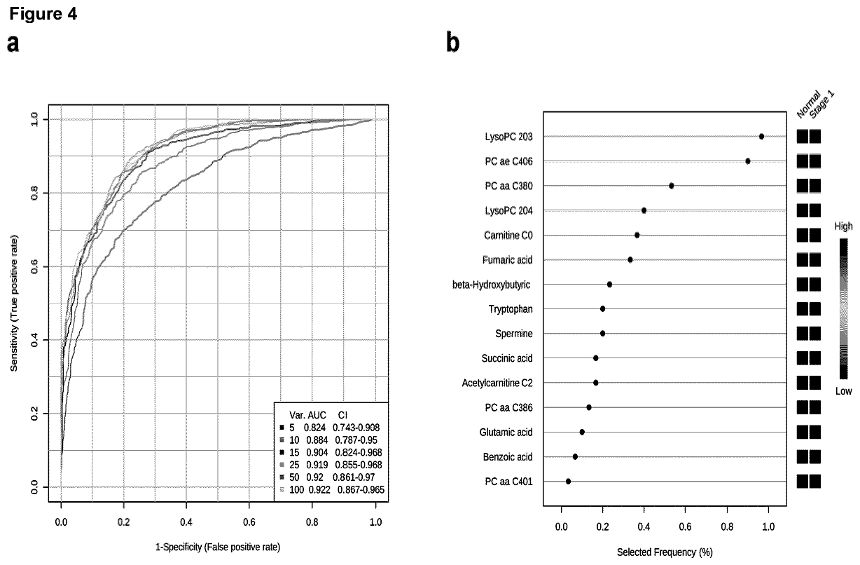

Logistic regression along with random forest based exploratory ROC analysis was performed using MetaboAnalyst to identify the best metabolite combination to distinguish stage I NSCLC from healthy controls. In this analysis, balanced sub-sampling-based Monte-Carlo cross validation (MCCV) was used to generate the receiver-operating characteristic (ROC) curves. Using a discovery cohort of plasma samples from 40 healthy controls and 47 stage I NSCLC patients, the AUC of different ROC models with different numbers of metabolite features ranged from 0.824 to 0.922 (FIG. 3a). FIG. 3b shows the most frequently selected metabolites with LysoPC 20:3, PC ae C40:6, PC aa C38:0, LysoPC 20:4, fumaric acid, carnitine, and β-hydroxybutyric acid being identified as the top-listed metabolites. A logistic regression model was then built to predict the probability of having stage I NSCLC (P) with the following equation: log(P/(1−P))=0.258−1.341×PC ae C40:6+1.747×LysoPC 20:3+0.913×β-hydroxybutyric acid+0.939×Fumaric acid, where the concentration of each named metabolite in the equation is given in μM. The ROC curve with 95% confidence interval (CI) is shown in FIG. 3a. The AUC and the 10-fold cross-validation AUC of the ROC curve was 0.931 (95% CI, 0.924˜0.955) and 0.923 (95% CI, 0.866˜0.980), respectively. The performance of the metabolites-only model was further checked on the validation set (which consisted of 20 healthy controls and 23 stage I cancer patients) and a slightly lower AUC was obtained (0.890). The ROC curve obtained from the validation set is shown in FIG. 3a as well. Other details of the model are listed in Table 4 below.

TABLE 4
Logistic regression based optimal model for
stage I NSCLC detection: metabolites only.
Logistic Model with Selected Compounds:
log(P/(1 − P)) = 0.258 − 1.341 × PC ae C40:6 + 1.747 × LysoPC 20:3 +
0.913 × β-hydroxybutyric acid + 0.939 × Fumaric acid (“Formula 1”)
The optimal cut-off point for the above equation is 0.69.
Logistic Regression Model - Summary of Each Feature:

| | Estimate | Std. Error | z value | Pr(>\|z\|) | Odds |
|---|---|---|---|---|---|
| (Intercept) | 0.258 | 0.352 | 0.733 | 0.463 | — |
| LysoPC 20:3 | 1.747 | 0.518 | 3.37 | 0.001 | 5.73 |
| β-Hydroxybutyric acid | 0.913 | 0.404 | 2.263 | 0.024 | 2.49 |
| Fumaric acid | 0.939 | 0.446 | 2.106 | 0.035 | 2.56 |
| PC ae C40:6 | −1.341 | 0.465 | −2.884 | 0.001 | 0.26 |

Performance of Logistic Regression Model:

| | AUC | Sensitivity | Specificity |
|---|---|---|---|
| Training/Discovery | 0.939 (0.924~0.955) | 0.827 (0.791~0.863) | 0.957 (0.936~0.977) |
| 10-fold Cross- | 0.923 (0.866~0.980) | 0.830 (0.830~0.937) | 0.927 (0.847~1.000) |
| Validation | | | |

When the smoking history of patients was added, the logistic model for the discovery cohort was modified to logit(P)=log(P/(1−P))=0.311+0.641×Amount of Smoking−1.372×PC ae C40:6+1.623×LysoPC 20:3+0.882×β-hydroxybutyric acid+0.65×Fumaric acid, where P is the probability of stage I NSCLC. As before, the concentration of each named metabolite in the equation is given in μM. Here and in all other models below, the Amount of Smoking was calculated by multiplying the period of smoking (in days) by the daily amount of smoking (cig/day). The ROC curve of the corresponding model is shown in FIG. 3b. The AUC for the metabolite+smoking model was 0.942 (95% CI, 0.926˜0.957) and after 10-fold cross-validation it was 0.922 (95% CI, 0.864˜0.979). This was similar to the metabolite-only model. When the same metabolite+smoking history model was tested on the validation set, the AUC of the validation cohort was essentially the same (0.920, FIG. 3b) as the metabolite-only model. Interestingly, the sensitivity of the model was modestly increased when smoking history was taken into consideration (Table 5 below).

TABLE 5
Logistic regression based optimal model for stage I
NSCLC detection: metabolites plus smoking history.
Logistic Model with Selected Compounds:
logit(P) = log(P/(1 − P)) = 0.311 + 0.641 × Amount of smoking −
1.372 × PC ae C40:6 + 1.623 × LysoPC 20:3 + 0.882 × β-
hydroxybutyric acid + 0.65 × Fumaric acid (“Formula 2”)
The optimal cut-off point for the above equation is 0.74.
Logistic Regression Model - Summary of Each Feature:

| | Estimate | Std. Error | z value | Pr(>\|z\|) | Odds |
|---|---|---|---|---|---|
| (Intercept) | 0.311 | 0.369 | 0.843 | 0.399 | — |
| Amount of smoking | 0.641 | 0.382 | 1.676 | 0.094 | 1.9 |
| PC ae C40:6 | −1.372 | 0.475 | −2.886 | 0.004 | 0.25 |
| LysoPC 20:3 | 1.623 | 0.495 | 3.281 | 0.001 | 5.07 |
| β-Hydroxybutyric acid | 0.882 | 0.419 | 2.105 | 0.035 | 2.42 |
| Fumaric acid | 0.65 | 0.474 | 1.373 | 0.17 | 1.92 |

Performance of Logistic Regression Model:

| | AUC | Sensitivity | Specificity |
|---|---|---|---|
| Training/Discovery | 0.942 (0.926~0.957) | 0.844 (0.809~0.879) | 0.951 (0.929~0.973) |
| 10-fold Cross- | 0.922 (0.864~0.979) | 0.851 (0.851~0.953) | 0.951 (0.882~1.000) |
| Validation | | | |

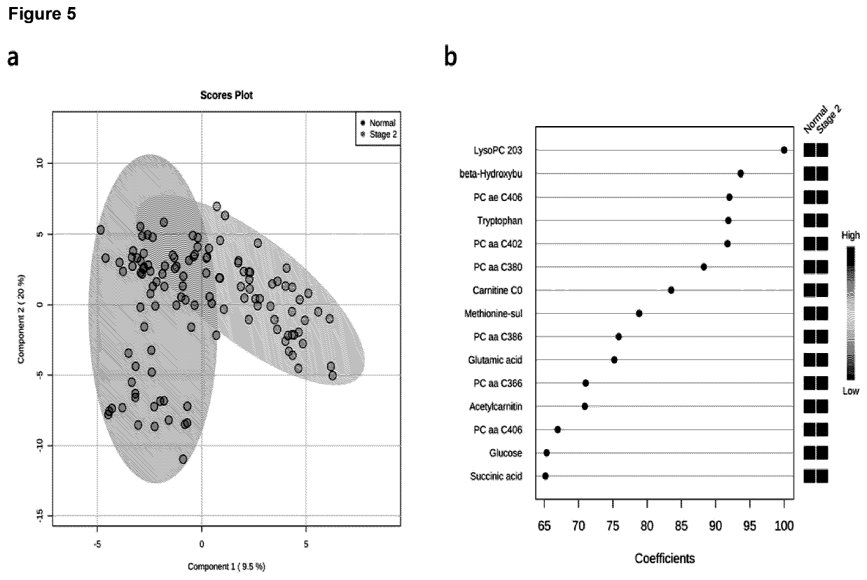

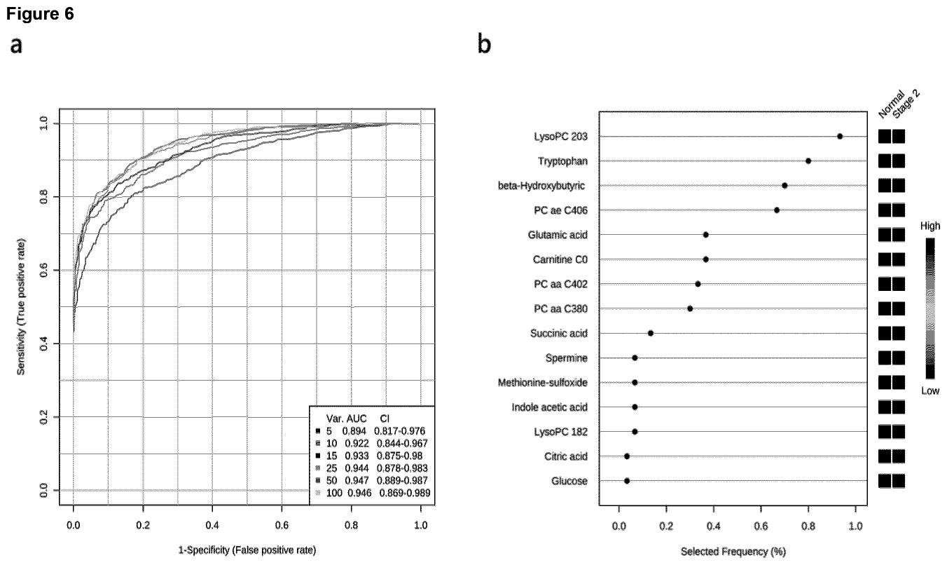

A similar series of analyses was carried out for lung cancer patients at stage II. The corresponding PLS-DA plot along with the VIP plot are shown in FIGS. 5a and 5b. Permutation testing revealed that the observed separation of the cases from the normal group was not due to chance (p-value<0.001). Comparing with NSCLC patients at stage I, fumaric acid was no longer identified as one of the most important features in the PLS-DA VIP plot, while β-hydroxybutyric acid was identified as one of the metabolites with the highest coefficient score.

Using a discovery cohort of plasma samples consisting of 40 healthy controls and 40 stage II NSCLC patients, the AUC of different metabolite-only regression models with different numbers of metabolite features ranged from 0.894 to 0.946 (FIG. 5a). FIG. 5b shows the most frequently selected metabolites. LysoPC 20:3, tryptophan, 3-hydroxybutyric acid, PC ae C40:6, glutamic acid, and carnitine were identified as the most differentiating metabolites.

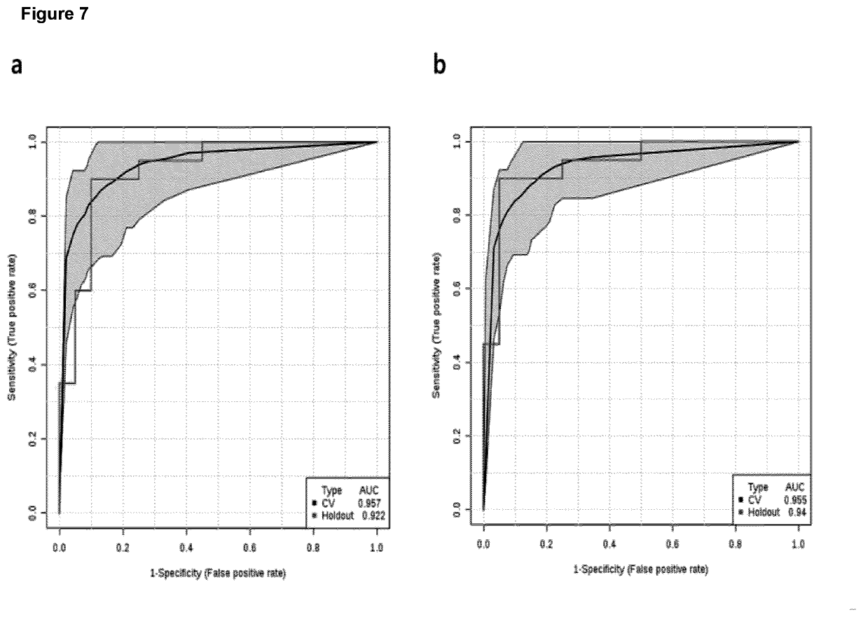

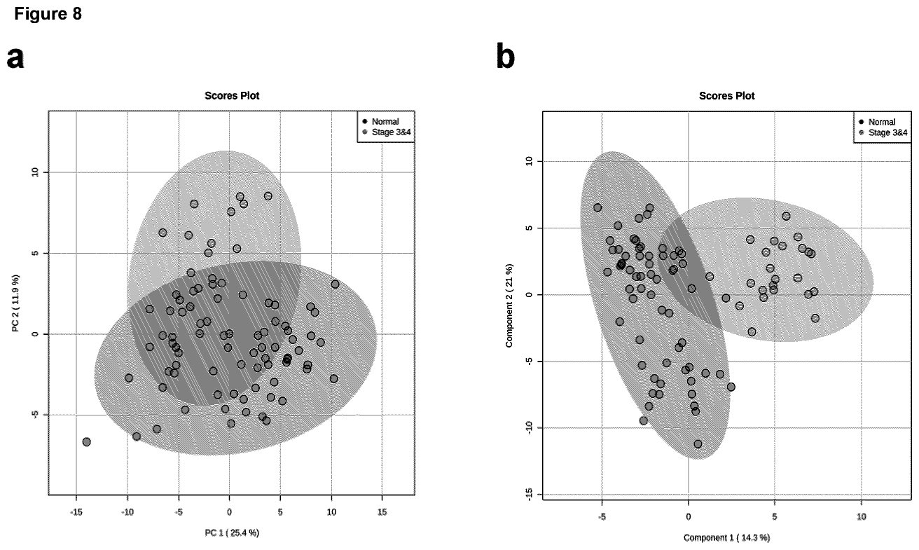

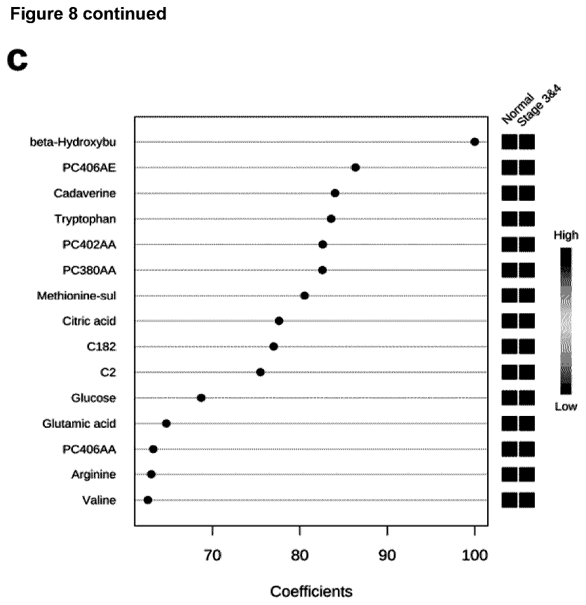

A logistic regression model was then built to predict the probability of having stage II NSCLC (P) with the following equation: logit(P)=log(P/(1−P))=0.346+2.565×β-hydroxybutyric acid−2.219×Citric acid+2.904×Carnitine−1.599×PC ae C40:6, where the concentration of each named metabolite in the equation is given in μM. The ROC curve with its 95% CI is shown in FIG. 7a. The AUC and the 10-fold cross-validation AUC of the ROC curve is 0.980 (95% CI, 0.973˜0.987) and 0.952 (95% CI, 0.909˜0.995), respectively. The performance of the metabolite-only model was further checked on the holdout validation set (which consisted of 20 healthy controls and 20 stage II cancer patients) and a slightly lower AUC was obtained (0.922). The ROC curve obtained from the validation set is shown in FIG. 7a as well. Other details of the model are listed in Table 6 below.

TABLE 6
Logistic regression based optimal model for
stage II NSCLC detection: metabolites only.
Logistic Model with Selected Compounds:
logit(P) = log(P/(1 − P)) = 0.346 + 2.565 × β-hydroxybutyric acid − 2.219 ×
Citric acid + 2.904 × Carnitine − 1.599 × PC ae C40:6. (“Formula 3”)
The optimal cut-off point for the above equation is 0.34.
Logistic Regression Model - Summary of Each Feature:

| | Estimate | Std. Error | z value | Pr(>\|z\|) | Odds |
|---|---|---|---|---|---|
| (Intercept) | 0.346 | 0.516 | 0.671 | 0.502 | — |
| β-Hydroxybutyric acid | 2.565 | 0.861 | 2.981 | 0.003 | 13.93 |
| Citric acid | −2.219 | 0.804 | −2.758 | 0.006 | 0.11 |
| Carnitine | 2.904 | 0.976 | 2.975 | 0.003 | 18.24 |
| PC ae C40:6 | −1.599 | 0.765 | −2.091 | 0.037 | 0.2 |

Performance of Logistic Regression Model:

| | AUC | Sensitivity | Specificity |
|---|---|---|---|
| Training/Discovery | 0.980 (0.973~0.987) | 0.958 (0.938~0.979) | 0.881 (0.854~0.909) |
| 10-fold Cross- | 0.952 (0.909~0.995) | 0.875 (0.875~0.977) | 0.875 (0.773~0.977) |
| Validation | | | |

When the smoking history of patients was added, the logistic model for the discovery cohort was modified to logit(P)=log(P/(1−P))=0.098+1.489×Amount of smoking+2.911×β-hydroxybutyric acid−1.627×Citric acid+2.605×Carnitine−0.702×PC ae C40:6, where P is the probability of stage II NSCLC and the concentration of each named metabolite in the equation is given in μM. The ROC curve of the corresponding model is shown in FIG. 7b. The AUC of the ROC curve for the metabolite+smoking model was 0.985 (95% CI, 0.979˜0.991) and after 10-fold cross-validation it was 0.948 (9500 CI, 0.900˜0.996). When the same metabolite+smoking history model was tested on the validation set, AUC of the validation set was also close to the training set (0.940, FIG. 7b). Similar to the model for stage I NSCLC, the sensitivity of the model and the overall model performance on the validation set was improved when smoking history was taken into consideration (Table 7 below).

TABLE 7
Logistic regression based optimal model for stage II
NSCLC detection: metabolites plus smoking history.
Logistic Model with Selected Compounds:
logit(P) = log(P/(1 − P)) = 0.098 + 1.489 × Amount of smoking +
2.911 × β-hydroxybutyric acid − 1.627 × Citric acid +
2.605 × Carnitine − 0.702 × PC ae C40:6 (“Formula 4”)
The optimal cut-off point for the above equation is 0.25.
Logistic Regression Model - Summary of Each Feature:

| | Estimate | Std. Error | z value | Pr(>\|z\|) | Odds |
|---|---|---|---|---|---|
| (Intercept) | −0.098 | 0.612 | 0.159 | 0.873 | — |
| Amount of smoking | 1.489 | 0.915 | 1.627 | 0.104 | 4.43 |
| β-Hydroxybutyric acid | 2.911 | 1.132 | 2.572 | 0.01 | 18.37 |
| Citric acid | −1.627 | 0.864 | −1.883 | 0.06 | 0.2 |
| Carnitine | 2.605 | 0.936 | 2.784 | 0.005 | 13.53 |
| PC ae C40:6 | −0.702 | 0.862 | −0.814 | 0.416 | 0.5 |

Performance of Logistic Regression Model:

| | AUC | Sensitivity | Specificity |
|---|---|---|---|
| Training/Discovery | 0.985 (0.979~0.991) | 0.972 (0.955~0.989) | 0.875 (0.841~0.909) |
| 10-fold Cross- | 0.948 (0.900~0.996) | 0.925 (0.925~1.000) | 0.850 (0.739~0.961) |
| Validation | | | |

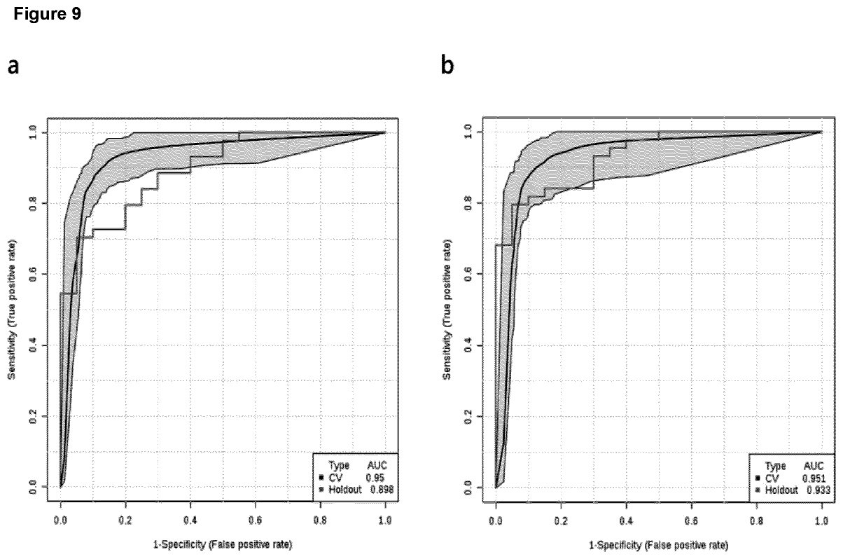

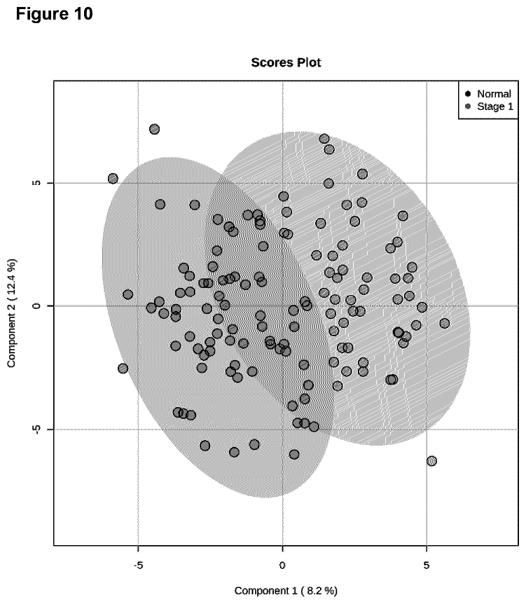

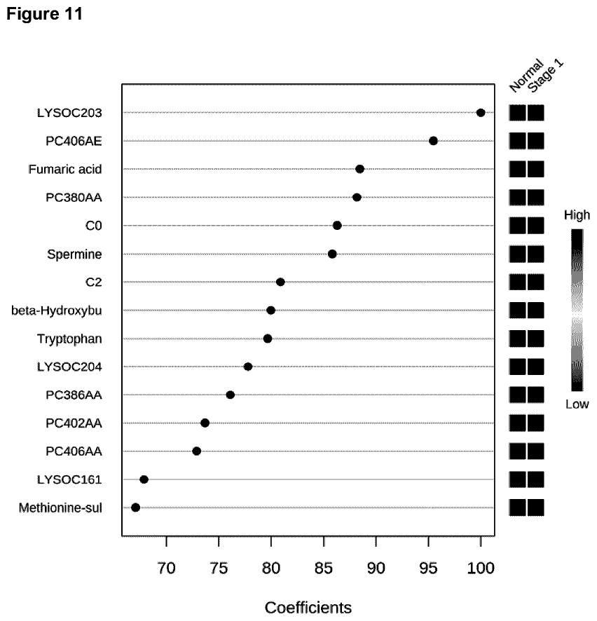

The same methods described above were applied to obtain a predictive model for diagnosing stage I+II NSCLC patients together (defined as early stage NSCLC). Using a discovery cohort of plasma samples from 40 healthy controls and 87 early stage NSCLC patients, a logistic regression model was built to predict the probability of having early stage NSCLC (P) with the following equation: logit(P)=log(P/(1−P))=2.346−1.528×PC ae C40:6+1.429×β-hydroxybutyric acid−2.481×Citric acid+1.03×LysoPC 20:3+1.773×Fumaric acid, where the concentration of each named metabolite in the equation is given in μM. The ROC curve with its 95% CI is shown in FIG. 9a. The AUC and the 10-fold cross-validation AUC of the ROC curve was 0.974 (95% CI, 0.965˜0.982) and 0.959 (95% CI, 0.923˜0.995), respectively. The performance of the metabolite-only model was further checked on the validation set (which consisted of 20 healthy controls and 43 early-stage patients) and a slightly lower AUC was obtained (0.898). The ROC curve obtained from the validation set and other details of the model are shown in FIG. 9a and Table 8 (below), respectively.

TABLE 8
Logistic regression based optimal model for stages
I + II NSCLC detection: metabolites only.
Logistic Model with Selected Compounds:
logit(P) = log(P/(1 − P)) = 2.346 − 1.528 × PC ae C40:6 +
1.429 × β-hydroxybutyric acid − 2.481 × Citric acid + 1.03 ×
LysoPC 20:3 + 1.773 × Fumaric acid. (“Formula 5”)
The optimal cut-off point for the above equation is 0.62.
Logistic Regression Model - Summary of Each Feature:

| | Estimate | Std. Error | z value | Pr(>\|z\|) | Odds |
|---|---|---|---|---|---|
| (Intercept) | 2.346 | 0.588 | 3.991 | <0.001 | — |
| PC ae C40:6 | −1.528 | 0.61 | −2.507 | 0.012 | 0.22 |
| β-Hydroxybutyric acid | 1.429 | 0.505 | 2.832 | 0.005 | 4.18 |
| Citric acid | −2.481 | 0.642 | −3.863 | <0.001 | 0.08 |
| LysoPC 20:3 | 1.03 | 0.508 | 2.028 | 0.043 | 2.8 |
| Fumaric acid | 1.773 | 0.569 | 3.117 | 0.002 | 5.89 |

Performance of Logistic Regression Model:

| | AUC | Sensitivity | Specificity |
|---|---|---|---|
| Training/Discovery | 0.974 (0.965~0.982) | 0.937 (0.920~0.954) | 0.922 (0.895~0.950) |
| 10-fold Cross- | 0.959 (0.923~0.995) | 0.919 (0.919~0.976) | 0.900 (0.807~0.993) |
| Validation | | | |

When the smoking history of patients was added, the logistic model for the discovery cohort was modified to logit(P)=log(P/(1−P))=2.427+1.425×Amount of smoking−1.414×PC ae C40:6+1.414×β-hydroxybutyric acid−2.193×Citric acid+1.738×LysoPC 20:3+1.44×Fumaric acid, where P is the probability of stage II NSCLC and the concentration of each named metabolite in the equation is given in μM. The ROC curve of the corresponding model is shown in FIG. 5b. The AUC of the ROC curve for the metabolite+smoking model was 0.982 (950 CI, 0.975˜0.990) and after 10-fold cross-validation it was 0.948 (95 CI, 0.930˜1.000). When the same metabolite+smoking history model was tested on the validation set, the AUC of the validation set was reasonably close to the training set (0.933, FIG. 5b). Again, when smoking history was added into the model, both the sensitivities/specificities of the model and model performance were improved (Table 9 below).

TABLE 9
Logistic regression based optimal model for stages I +
II NSCLC detection: metabolites plus smoking history.
Logistic Model with Selected Compounds:
logit(P) = log(P/(1 − P)) = 2.427 + 1.425 × Amount of smoking − 1.414 ×
PC ae C40:6 + 1.414 × β-hydroxybutyric acid − 2.193 × Citric acid +
1.738 × LysoPC 20:3 + 1.44 × Fumaric acid (“Formula 6”)
The optimal cut-off point for the above equation is 0.66.
Logistic Regression Model - Summary of Each Feature:

| | Estimate | Std. Error | z value | Pr(>\|z\|) | Odds |
|---|---|---|---|---|---|
| (Intercept) | 2.427 | 0.638 | 3.803 | <0.001 | — |
| Amount of smoking | 1.425 | 0.507 | 2.813 | 0.005 | 4.16 |
| PC ae C40:6 | −1.048 | 0.64 | −1.637 | 0.102 | 0.35 |
| β-Hydroxybutyric acid | 1.414 | 0.594 | 2.379 | 0.017 | 4.11 |
| Citric acid | −2.193 | 0.719 | −3.051 | 0.002 | 0.11 |
| LysoPC 20:3 | 1.738 | 0.739 | 2.351 | 0.019 | 5.68 |
| Fumaric acid | 1.44 | 0.612 | 2.352 | 0.019 | 4.22 |

Performance of Logistic Regression Model:

| | AUC | Sensitivity | Specificity |
|---|---|---|---|
| Training/Discovery | 0.982 (0.975~0.990) | 0.960 (0.946~0.974) | 0.944 (0.921~0.968) |
| 10-fold Cross- | 0.965 (0.930~1.000) | 0.930 (0.930~0.984) | 0.925 (0.843~1.000) |
| Validation | | | |

Metabolite analysis of the plasma of patients at advanced stages of NSCLC were much more distinct from healthy controls, compared with earlier NSCLC stages. Both PCA and PLS-DA responded clear separation (Figure S4a and Figure S4b). The VIP data from the PLS-DA analysis showed that ketone body dysregulation appeared to be one of the most characteristic features of stage IIIB+IV NSCLC patients (Figure S4c). Elevated levels of cadaverine, a product of lysine decarboxylation, was also identified as one of the most important features in discriminating stages IIIB+IV NSCLC. In contrast, upregulation of LysoPC20:3, which was a feature of stage I/II NSCLC did not stand out as an important feature in stage III/IV NSCLC. As the identification of markers for late stage lung cancer was not a major focus of this work (and because of the relatively small sample size), a logistic regression model to predict stage IIIB/IV NSCLC was not developed.

The purpose of this study was to discover and validate a combination of plasma metabolite (and clinical) biomarkers for the early detection of non-small cell lung cancer (NSCLC). In particular, plasma metabolite changes in NSCLC patients (at various stages) versus healthy (age and gender-matched) controls were studied via quantitative MS-based metabolomic techniques. Separate discovery cohorts (with 10-fold cross validation) and validation cohorts were used to prevent overtraining and any unintended bias in the results. Three different metabolite-only and three different metabolite+smoking status models were developed and independently validated to detect stage I, stage II and stage I/II NSCLC. Most of these models achieved AUCs>0.9.

A key advantage of developing a blood-based metabolomic test is that it can be easily converted into a low-cost, high-throughput assay that can be run at almost all clinical laboratories equipped with standard triple-quadrupole mass spectrometers. A modified assay that is specific to the metabolites identified here may be run at a rate of 4-5 minutes per sample using as little as 10 μL of plasma. These promising results suggest that a minimally invasive, high performance, high-throughput, low cost lung cancer screening assay might be developed that could be used to select patients for further follow-up and confirmation using LDCT or other lung imaging modalities.

Accordingly, the skilled person understands that this disclosure relates to a method and, in particular embodiments, a method of detecting non-small cell lung cancer (e.g. stage I or stage II non-small cell lung cancer). The method comprises determining the concentration of each metabolite of a group of metabolites in a biological sample from a subject, wherein the group of metabolites comprises: β-hydroxybutyric acid, LysoPC 20:3, PC ae C40:6, citric acid, carnitine, and fumaric acid; β-hydroxybutyric acid, LysoPC 20:3, PC ae C40:6, and fumaric acid; or β-hydroxybutyric acid, PC ae C40:6, citric acid, and carnitine.

In various embodiments, the group of metabolites comprises β-hydroxybutyric acid, LysoPC 20:3, PC ae C40:6, and fumaric acid. In various embodiments, the group of metabolites consists essentially of β-hydroxybutyric acid, LysoPC 20:3, PC ae C40:6, and fumaric acid. In such embodiments, the method comprises determining a probability score for the biological sample according to the formula 1:

logit(P)=log(P/(1−P))=0.258−1.341×PC ae C40:6+1.747×LysoPC 20:3+0.913×β-hydroxybutyric acid+0.939×fumaric acid (formula 1)

The numeric value of each metabolite in the equation is the concentration in uM of the metabolites after median normalization, log transformation and auto-scaling. A probability score that meets or exceeds a stage I threshold indicates that the subject has stage I non-small cell lung cancer.

In other embodiments, the subject is a smoker. In such embodiments, the method comprises determining a probability score for the biological sample according to the formula 2:

logit(P)=log(P/(1−P))=0.311+0.641×Amount of Smoking−1.372×PC ae C40:6+1.623×LysoPC 20:3+0.882×β-hydroxybutyric acid+0.65×fumaric acid (formula 2).

The numeric value of each metabolite in the equation is the concentration in uM of the metabolites after median normalization, log transformation and auto-scaling. A probability score that meets or exceeds a stage I smoker threshold indicates that the subject has stage I non-small cell lung cancer.

In various embodiments, the group of metabolites comprises: β-hydroxybutyric acid; PC ae C40:6; citric acid; and carnitine. In some embodiments, the group of metabolites consists essentially of β-hydroxybutyric acid, PC ae C40:6, citric acid, and carnitine. In such embodiments, particularly, where the subject is a non-smoker, the method comprises determining a stage I probability score for the biological sample according to the formula 3:

logit(P)=log(P/(1−P))=0.346+2.565×β-hydroxybutyric acid−2.219×citric acid+2.904×carnitine−1.599×PC ae C40:6; (formula 3).

The numeric value of each metabolite in the equation is the concentration in uM of the metabolites after median normalization, log transformation and auto-scaling. A probability score that meets or exceeds a stage II threshold indicates that the subject has stage II non-small cell lung cancer.

In other embodiments, the subject is a smoker. In such embodiments, the method comprises determining a stage I probability score for the biological sample according to the formula 4:

logit(P)=log(P/(1−P))=0.098+1.489×Amount of Smoking+2.911×β-hydroxybutyric acid−1.627×citric acid+2.605×Carnitine−0.702×PC ae C40:6 (formula 4).

The numeric value of each metabolite in the equation is the concentration in uM of the metabolites after median normalization, log transformation and auto-scaling. A probability score that meets or exceeds a stage II smoker threshold indicates that the subject has stage II non-small cell lung cancer.

In other embodiments, the group of metabolites comprises: β-hydroxybutyric acid; LysoPC 20:3; PC ae C40:6; citric acid; and fumaric acid. In various embodiments, the group of metabolites consists essentially of β-hydroxybutyric acid, LysoPC 20:3, PC ae C40:6, citric acid, and fumaric acid. In such embodiments, particularly where the subject is a non-smoker, the method comprises determining a probability score for the biological sample according to the formula 5:

logit(P)=log(P/(1−P))=2.346−1.528×PC ae C40:6+1.429×β-hydroxybutyric acid−2.481×citric acid+1.03×LysoPC 20:3+1.773×fumaric acid;

The numeric value of each metabolite in the equation is the concentration in uM of the metabolites after median normalization, log transformation and auto-scaling. A probability score that meets or exceeds a stage I/II probability threshold indicates that the subject has stage I or state II non-small cell lung cancer.

In other embodiments where the subject is a smoker, the method comprises determining a probability score for the biological sample according to the formula 6:

logit(P)=log(P/(1−P))=2.427+1.425×Amount of Smoking−1.414×PC ae C40:6+1.414×βhydroxybutyric acid−2.193×citric acid+1.738×LysoPC 20:3+1.44×fumaric acid (formula 6).

The numeric value of each metabolite in the equation is the concentration in uM of the metabolites after median normalization, log transformation and auto-scaling. A probability score that meets or exceeds a stage I/II probability threshold indicates that the subject has stage I or state II non-small cell lung cancer.

In various embodiments, the group of metabolites consists essentially of β-hydroxybutyric acid, LysoPC 20:3, PC ae C40:6, citric acid, carnitine, and fumaric acid. The skilled person understands that, in such embodiments involving all six of these metabolites, the subject can be analyzed for likelihood of stage I and stage II non-small cell lung cancer according with each of the formulae, perhaps simultaneously. In such embodiments, particularly where the subject is a non-smoker, the method comprises determining a stage I probability score for the biological sample according to formula 1. A stage I probability score that meets or exceeds a stage I threshold for formula 1 indicates that the subject has stage I non-small cell lung cancer.

At the same time, the method may further include determining a stage II probability score for the biological sample according to the formula 3. A stage II probability score that meets or exceeds a stage II threshold for formula 3 indicates that the subject has stage II non-small cell lung cancer.

Yet still at the same time, the method may further comprise determining a stage I/II probability score for the biological sample according to the formula 5. A stage I/II probability score that meets or exceeds a stage I/II threshold indicates that the subject has stage I or stage II non-small cell lung cancer.

In embodiments where the subject is a smoker, the method may comprise determining a stage I probability score for the biological sample according to the formula 2. A stage I probability score that meets or exceeds a stage I threshold indicates that the subject has stage I non-small cell lung cancer.

At the same time, the method may further include determining determining a stage II probability score for the biological sample according to the formula 4. A stage II probability score that meets or exceeds a stage II threshold for formula 4 indicates that the subject has stage II non-small cell lung cancer.

Yet still at the same time, the method may further comprise determining a stage I/II probability score for the biological sample according to the formula 6. A stage I/II probability score that meets or exceeds a stage I/II threshold for formula 6 indicates that the subject has stage I or stage II non-small cell lung cancer.

Of course, the skilled person understands that, when the concentrations all 6 of the metabolites are determined, the analysis according to formula 1, 3, and 5 (or 2, 4, and 6 if the subject are smokers) may be carried out in any order. Alternatively, only 1 or 3 of the analyses may be conducted.

### Detection of Cancer Using LYSO-PC 20:3 (a Lysophospholipid), β-Hydroxybutyric Acid, Fumaric Acid and Spermine

This disclosure also relates to a set of four serum metabolite biomarkers for early lung cancer diagnosis that exhibit AUROCs (Area Under the Receiver Operating Characteristic curve) of 0.94 for stage I lung cancer with a specificity of 84% and a sensitivity of 90%. When combined with easily measured clinical data, namely, past smoking history and amount of smoking, the AUROC for stage I lung cancer increased slightly to 0.95 with a sensitivity and specificity of 91% and 92%, respectively. This is may be among the highest AUROC's reported for any test for lung cancer, regardless of staging. The four serum markers are LYSO-PC 20:3 (a lysophospholipid), β-hydroxybutyric acid, fumaric acid and spermine.

A metabolomic analysis of 216 serum samples by liquid chromatography-mass spectrometry (LC-MS) was performed on lung cancer patients (n=156) and healthy controls (n=60). The lung cancer patient group included seventy patients with stage I lung cancer, sixty patients with stage II cancer and twenty-six patients with stage II/IV cancer. All lung cancer patients were identified as having non-small-cell lung carcinoma (NSCLC) which is the most common form of lung cancer.

The targeted LC-MS study was performed using the TMIC-Prime™ assay a targeted, quantitative metabolomic assay kit developed and extensively validated by The Metabolomics Innovation Center (TMIC) of BSB Z-824, Department of Biological Sciences, University of Alberta, Edmonton, Alberta, Canada T6G 2R3. The TMIC-Prime™ assay measures one hundred and forty-three different endogenous metabolites including amino acids, acylcarnitines, organic acids, biogenic amines, uremic toxins, glycerophospholipids, sphingolipids and sugars. The TMIC-Prime™ assay uses a combination of direct injection mass spectrometry and a reverse-phase LC-MS/MS custom assay optimized for an ABI 4000 Q-Trap available from Applied Biosystems/MDS Sciex mass spectrometer equipped with an Agilent 1100 series HPLC system. The method combines the derivatization and extraction of analytes, and selective MS detection using multiple reaction monitoring (MRM) pairs. Isotopically-labeled internal standards are used for metabolite quantification.

The custom assay contains a 96 deep-well plate with a filter plate attached with sealing tape, along with all the reagents and solvents used to prepare the plate assay. The first 14 wells of each plate are used for quality control (QC) and instrument calibration and consist of one blank, three “zero” samples, seven calibration standards and three quality control samples. For all metabolite measurements except the organic acid measurements, serum samples were thawed on ice, then vortexed and centrifuged at 13,000×g. 10 μL of each serum sample was loaded onto the center of the filter on the upper 96-well plate and dried in a stream of nitrogen. Subsequently, phenyl-isothiocyanate was added to derivative all amino-containing groups. After incubation, the filter spots were dried again using an evaporator. Extraction of the metabolites was then achieved by adding 300 μL of extraction solvent (MeOH and H₂O). The extracts were obtained by centrifugation into the lower 96-deep well plate, followed by a dilution step with an MS running solvent. For organic acid analysis, 150 μL of ice-cold methanol and 10 μL of isotope-labeled internal standard mixture was added to 50 μL of serum for overnight protein precipitation. The resulting sample was then centrifuged at 13000×g for 20 min. 50 μL of the supernatant was then loaded into the center of wells of a 96-deep well plate, followed by the addition of 3-nitrophenylhydrazine (NPH) for derivatization of the carboxylate groups. After incubation for 2 h, BHT stabilizer and water were added prior to LC-MS injection.

A total of one hundred and thirty-eight metabolites were quantitatively measured in each of the 216 serum samples in the LC-MS method. Statistical preprocessing removed 35 metabolites due to the fact that 20% of their MS signals were below the MS detection limit. To identify potentially diagnostic metabolites and generate lung cancer detection models, a series of statistical and computational procedures were performed as previously described in Wishart, D. S., (2010) Computational approaches to metabolomics. Methods Mol Biol. 593: 283-313. By applying a simple Student t-test to our metabolomics data set, significant differences between the metabolic profiles of healthy controls and lung cancer patients (all stages) were revealed. Multivariate statistics and logistic regression analyses were carried out to discover a minimum-sized metabolite panel needed to accurately diagnose early stage NSCLC. Partial least squares discriminant analysis (PLS-DA) was performed using MetaboAnalyst as disclosed in Xia, J., et al., (2015) MetaboAnalyst 3.0—making metabolomics more meaningful. Nucleic Acids Res. 43(W1): W251-W257. This led to good separation between NSCLC patients and healthy controls. Permutation testing demonstrated that the observed separation was statistically significant (p<0.001). Biomarker metabolite panels predictive of NSCLC were identified using logistic regression modeling with a Lasso algorithm. This method was also used to analyze the correlation of clinical parameters with NSCLC. The resulting models were ranked according to their AUROC value (high to low). Using this protocol, we were able to identify metabolite biomarkers that could distinguish early stage lung cancer, i.e. patients with stage I lung cancer, from healthy controls with AUROC values above 0.90. 10-fold cross-validation was applied to validate the models. Sensitivity and specificity was calculated from the ROC curve with a 95% confidence interval in both the training and validation steps of building the model.

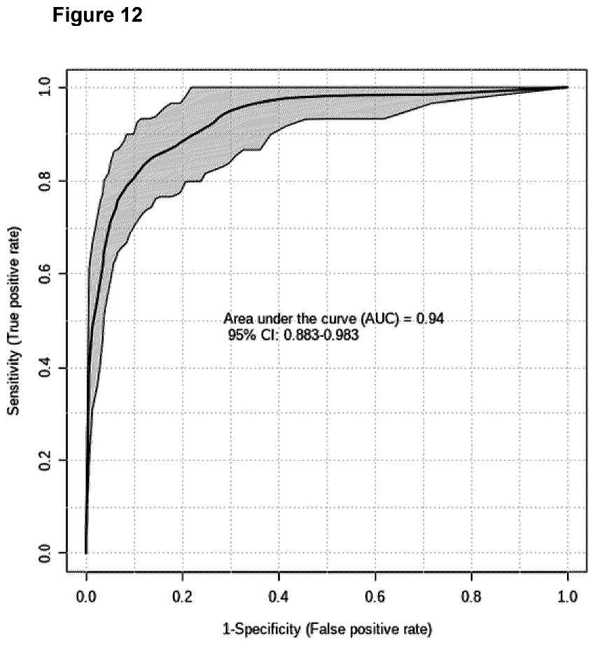

FIG. 10 shows the PLS-DA analysis which resulted in a detectable separation between lung cancer patients with stage I lung cancer (shown in in shaded area on right) compared to healthy controls (shown in shaded area on left). FIG. 11 displays the VIP plot. Permutation testing revealed that the observed separation of the cases from the normal group was highly unlikely to be due to chance (P<0.001). The resulting model for diagnosing stage I lung cancer consists of four serum metabolites as shown in a ROC curve in FIG. 12. The model is based on the level of LYSO-PC 20:3, β-hydroxybutyric acid, fumaric acid and spermine, and is represented by the probability of stage I NSCLC where (P) is log(P/(1−P)=0.504+2.192*LYSO-PC20:3+1.252*β-hydroxybutyric acid+1.23*fumaric acid−1.798*spermine. The AUROC value of the training set and the 10-fold cross-validated set is 0.95 (95% CI, 0.94˜0.96) and 0.94 (95% CI, 0.90˜0.98), respectively. The sensitivity and specificity with validation is 0.84 and 0.90, respectively. These metrics indicate that the model is a highly significant predictor of stage 1 NSCLC. Details of this stage I model are listed in Table 10. A similar analysis was performed for diagnosing stage II lung cancer and resulted in a similar set of metabolites and similar AUROC values as in diagnosing stage I lung cancer.

TABLE 10
Details of a logistic regression model to diagnose stage I lung cancer.
Logistic Model with Selected Compounds:
log(P/(1 − P) = 0.504 + 2.192*LYSO-PC20:3 + 1.252* β-
hydroxybutyric acid + 1.23*fumaric acid − 1.798*spermine.
Logistic Regression Model - Summary of Each Feature:

| | Estimate | Std. Error | z value | Pr(>\|z\|) | Odds |
|---|---|---|---|---|---|
| (Intercept) | 2.278 | 0.325 | 5.686 | <0.121 | — |
| β-Hydroxybutyric acid | 1.252 | 0.402 | 3.113 | 0.002 | 3.5 |
| Fumaric acid | 1.23 | 0.376 | 3.27 | 0.001 | 3.42 |
| Spermine | −1.798 | 0.434 | −4.147 | <0.001 | 0.17 |
| LYSO-PC20:3 | 2.192 | 0.535 | 4.093 | <0.001 | 2.31 |

Performance of Logistic Regression Model:

| | AUC | Sensitivity | Specificity |
|---|---|---|---|
| Training/Discovery | 0.950 (0.939~0.962) | 0.838 (0.809~0.867) | 0.917 (0.898~0.943) |
| 10-fold Cross- | 0.935 (0.895~0.976) | 0.843 (0.843~0.928) | 0.900 (0.824~0.976) |
| Validation | | | |

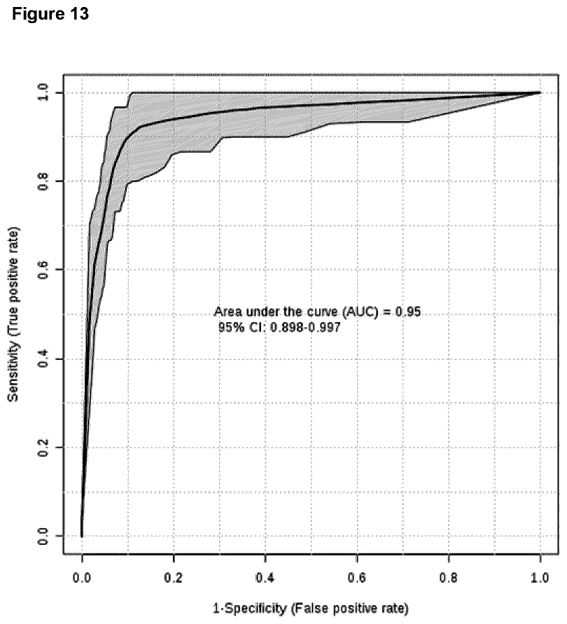

To improve the performance of the diagnostic models, the effect of multiple clinical variables, including age, gender, height, weight, and smoking history on lung cancer incidence rate were evaluated by logistic regression. Of these clinical parameters, only smoking history was identified as significantly related to lung cancer incidence rate (p-value=1.13*10⁻¹¹). A further logistic regression model between the lung cancer incidence rate and smoking history confirmed the significant positive correlation between former smokers and lung cancer incidence (p-value=4.16*10⁻¹⁰), with an odds ratio at 9.82. Our results also showed that current smokers had a significantly higher lung cancer incidence (p-value=7.082*10⁻¹¹). Although the correlation between smoking history and lung cancer has been heavily studied and widely accepted, our analysis revealed that the smoking history (including duration and amount of smoking) should be included in any diagnostic model for lung cancer as it improves the overall diagnostic performance. The ROC curve of the model that includes smoking history is shown in FIG. 13. The logistic model built with the four metabolites plus the period and amount of smoking is represented as log(P/(1−P)=0.739+0.68*fumaric acid−1.861*spermine+5.248*period of smoking−4.19*Cig/day+1.139*β-hydroxybutyric acid+1.776*LYSO-PC 20:3, where P is the probability of stage 1 NSCLC. The resulting AUROC from the training set is 0.96 (95% CI, 0.95˜0.97) and from 10-fold cross-validation is 0.95 (95% CI, 0.903˜0.985). The sensitivity and specificity from the validation set was 91% and 92%, respectively. Full details about the logistic regression model can be found in Table 11. The four metabolite biomarkers that we have identified for diagnosing stage I lung cancer are found in serum, allowing for a quick and simple blood-based test. Another advantage to our early stage lung cancer assay lies in the fact that it is a multi-component test. The advantage of using a multi-component biomarker panel is that it is possible to adjust the shape of the ROC curve to optimize sensitivity/specificity so as to greatly reduce the number of false negatives at the expense of increasing false positives (which is preferred for screening tests). The ROC curve shape adjustment is not possible with a single biomarker panel.

TABLE 11
Details of a logistic regression model to diagnose
stage I lung cancer including smoking history.
Logistic Model with Selected Compounds:
log(P/(1 − P) = 0.709 + 1.934*LYSO-PC20:3 + 1.142*β-hydroxybutyric
acid + 0.778*fumaric acid − 1.907*spermine + 1.37*Period of smoking.
Logistic Regression Model - Summary of Each Feature:

| | Estimate | Std. Error | z value | Pr(>\|z\|) | Odds |
|---|---|---|---|---|---|
| (Intercept) | 2.278 | 0.325 | 5.686 | <0.121 | — |
| β-Hydroxybutyric acid | 1.142 | 0.414 | 2.758 | 0.006 | 3.13 |
| Fumaric acid | 0.778 | 0.375 | 2.071 | 0.038 | 2.18 |
| Spermine | −1.907 | 0.494 | −3.863 | <0.001 | 0.15 |
| LYSO-PC20:3 | 1.934 | 0.511 | 3.783 | <0.001 | 6.92 |
| Period of smoking | 1.37 | 0.397 | 3.453 | 0.001 | 3.94 |

Performance of Logistic Regression Model:

| | AUC | Sensitivity | Specificity |
|---|---|---|---|
| Training/Discovery | 0.960 (0.949~0.971) | 0.924 (0.903~0.945) | 0.919 (0.895~0.942) |
| 10-fold Cross- | 0.950 (0.911~0.989) | 0.914 (0.914~0.980) | 0.917 (0.847~0.987) |
| Validation | | | |

Based on the knowledge described above, the skilled person will understand that aspects of this disclosure relates to a method which, in various aspects, may be a method of diagnosing non-small cell lung cancer. The method comprises determining the concentration of each metabolite of a group of metabolites in a biological sample from a subject, wherein the group of metabolites comprises β-hydroxybutyric acid, LysoPC 20:3, fumaric acid, and spermine. In various embodiments the group of metabolites consists of β-hydroxybutyric acid, LysoPC 20:3, fumaric acid, and spermine.

The method may further comprise determining a probability score for the biological sample according to the formula 7:

logit(P)=log(P/(1−P))=0.504+2.192×LysoPC 20:3+2.252×β-hydroxybutyric acid+1.23×fumaric acid−1.798×spermine (formula 7)

The numeric value of each metabolite in the equation is the concentration in uM of the metabolites after median normalization, log transformation and auto-scaling. A probability score that meets or exceeds a stage I threshold indicates that the subject has stage I non-small cell lung cancer. Such embodiments are particularly predictive for a subject that is a non-smoker.

In other embodiments, the subject may be a smoker. In such embodiments, the method further comprises determining a probability score for the biological sample according to the formula 8:

0.739+0.68×fumaric acid−1.861×spermine+5.248×period of smoking−4.19×Cig/day+1.139×β-hydroxybutyric acid+1.776×LYSO-PC 20:3; (formula 8)

The numeric value of each metabolite in the equation is the concentration in uM of the metabolites after median normalization, log transformation and auto-scaling.

A probability score that meets or exceeds a stage I threshold indicates that the subject has stage I non-small cell lung cancer.

### Treatment of Non-Small Cell Lung Cancer

The skilled person understands that, once a subject has been diagnosed as having stage I or stage II non-small cell lung cancer according to a method disclosed herein, then the subject may be treated according to treatment methods as are known in the art.

Treating the subject for lung cancer may include administering a therapeutic agent to the subject. The therapeutic agent may comprise various agents known or discovered to be useful for treating non-small cell lung cancer, including but not limited: Cisplatin; Carboplatin; Paclitaxel; Albumin-bound paclitaxel; Docetaxel; Gemcitabine; Vinorelbine; Etoposide; Pemetrexed; Bevacizumab; Ramucirumab; Erlotinib; Afatinib; Gefitinib; Osimertinib; Dacomitinib; Necitumumab; Crizotinib; Ceritinib; Lorlatinib; Entrectinib; Dabrafenib; Trametinib; Selpercatinib; pralsetinib; Capmatinib; Larotrectinib; entrectinib; Nivolumab; pembrolizumab; atezolizumab; Durvalumab; Ipilimumab; or combinations thereof.

Accordingly, the skilled person understands that aspects of this disclosure relate to use of a therapeutic agent to treat a subject diagnosed with non-small cell lung cancer according to a method as described herein. The therapeutic agent may included any agent know to be useful in treating non-small cell lung cancer, including by not limited to: Cisplatin; Carboplatin; Paclitaxel; Albumin-bound paclitaxel; Docetaxel; Gemcitabine; Vinorelbine; Etoposide; Pemetrexed; Bevacizumab; Ramucirumab; Erlotinib; Afatinib; Gefitinib; Osimertinib; Dacomitinib; Necitumumab; Crizotinib; Ceritinib; Lorlatinib; Entrectinib; Dabrafenib; Trametinib; Selpercatinib; pralsetinib; Capmatinib; Larotrectinib; entrectinib; Nivolumab; pembrolizumab; atezolizumab; Durvalumab; Ipilimumab; or combinations thereof.

It will be understood by a person skilled in the art that many of the details provided above are by way of example only, and are not intended to limit the scope of the invention which is to be determined with reference to the following claims.

What is claimed is:

1. A method, the method comprising determining the concentration of each metabolite of a group of metabolites in a biological sample from a subject, wherein the group of metabolites comprises: β-hydroxybutyric acid, LysoPC 20:3, PC ae C40:6, citric acid, carnitine, and fumaric acid; β-hydroxybutyric acid, LysoPC 20:3, PC ae C40:6, and fumaric acid; or β-hydroxybutyric acid, PC ae C40:6, citric acid, and carnitine.

2. The method of claim 1, wherein the group of metabolites comprises β-hydroxybutyric acid, LysoPC 20:3, PC ae C40:6, and fumaric acid.

3. The method of claim 1 or 3, wherein the group of metabolites consists essentially of β-hydroxybutyric acid, LysoPC 20:3, PC ae C40:6, and fumaric acid.

4. The method of claim 2 or 3, further comprising determining a probability score for the biological sample according to the formula 1:
logit(P)=log(P/(1−P))=0.258−1.341×PC ae C40:6+1.747×LysoPC 20:3+0.913×β-hydroxybutyric acid+0.939×fumaric acid;
wherein the numeric value of each metabolite in the equation is the concentration in uM of the metabolites after median normalization, log transformation and auto-scaling.

5. The method of claim 5, wherein a probability score that meets or exceeds a stage I threshold indicates that the subject has stage I non-small cell lung cancer.

6. The method of any one of claims 2 to 5, wherein the subject is a non-smoker.

7. The method of claim 2 or 3, wherein the subject is a smoker.

8. The method of claim 7, further comprising determining a probability score for the biological sample according to the formula 2:
logit(P)=log(P/(1−P))=0.311+0.641×Amount of Smoking−1.372×PC ae C40:6+1.623×LysoPC 20:3+0.882×β-hydroxybutyric acid+0.65×fumaric acid;
wherein the numeric value of each metabolite in the equation is the concentration in uM of the metabolites after median normalization, log transformation and auto-scaling.

9. The method of claim 8, wherein a probability score that meets or exceeds a stage I smoker threshold indicates that the subject has stage I non-small cell lung cancer.

10. The method of claim 1, wherein the group of metabolites comprises: β-hydroxybutyric acid; PC ae C40:6; citric acid; and carnitine.

11. The method of claim 10, wherein the group of metabolites consists essentially of β-hydroxybutyric acid, PC ae C40:6, citric acid, and carnitine.

12. The method of claim 10 or 11, further comprising determining a stage I probability score for the biological sample according to the formula 3:
logit(P)=log(P/(1−P))=0.346+2.565×β-hydroxybutyric acid−2.219×citric acid+2.904×carnitine−1.599×PC ae C40:6;
wherein the numeric value of each metabolite in the equation is the concentration in uM of the metabolites after median normalization, log transformation and auto-scaling.

13. The method of claim 12, wherein a probability score that meets or exceeds a stage II threshold indicates that the subject has stage II non-small cell lung cancer.

14. The method of any one of claims 10 to 13, wherein the subject is a non-smoker.

15. The method of claim 10 or 11, wherein the subject is a smoker.

16. The method of claim 15, further comprising determining a stage I probability score for the biological sample according to the formula 4:
logit(P)=log(P/(1−P))=0.098+1.489×Amount of Smoking+2.911×β-hydroxybutyric acid−1.627×citric acid+2.605×Carnitine−0.702×PC ae C40:6;
wherein the numeric value of each metabolite in the equation is the concentration in uM of the metabolites after median normalization, log transformation and auto-scaling.

17. The method of claim 16, wherein a probability score that meets or exceeds a stage II smoker threshold indicates that the subject has stage II non-small cell lung cancer.

18. The method of claim 1 or 2, wherein the group of metabolites comprises: β-hydroxybutyric acid; LysoPC 20:3; PC ae C40:6; citric acid; and fumaric acid.

19. The method of claim 18, wherein the group of metabolites consists essentially of β-hydroxybutyric acid, LysoPC 20:3, PC ae C40:6, citric acid, and fumaric acid.

20. The method of claim 18 or 19, further comprising determining a probability score for the biological sample according to the formula 5:
logit(P)=log(P/(1−P))=2.346−1.528×PC ae C40:6+1.429×β-hydroxybutyric acid−2.481×citric acid+1.03×LysoPC 20:3+1.773×fumaric acid;
wherein the numeric value of each metabolite in the equation is the concentration in uM of the metabolites after median normalization, log transformation and auto-scaling.

21. The method of claim 20, wherein a probability score that meets or exceeds a stage I/II probability threshold indicates that the subject has stage I or state II non-small cell lung cancer.

22. The method of any one of claims 18 to 21, wherein the subject is a non-smoker.

23. The method of claim 18 or 19, wherein the subject is a smoker.

24. The method of claim 23, further comprising determining a probability score for the biological sample according to the formula 6:
logit(P)=log(P/(1−P))=2.427+1.425×Amount of Smoking−1.414×PC ae C40:6+1.414×βhydroxybutyric acid−2.193×citric acid+1.738×LysoPC 20:3+1.44×fumaric acid;
wherein the numeric value of each metabolite in the equation is the concentration in uM of the metabolites after median normalization, log transformation and auto-scaling.

25. The method of claim 24, wherein a probability score that meets or exceeds a stage I/II probability threshold indicates that the subject has stage I or state II non-small cell lung cancer.

26. The method of claim 1, wherein the group of metabolites consists essentially of β-hydroxybutyric acid, LysoPC 20:3, PC ae C40:6, citric acid, carnitine, and fumaric acid.

27. The method of claim 1 or 26, further comprising determining a stage I probability score for the biological sample according to the formula 1:
logit(P)=log(P/(1−P))=0.258−1.341×PC ae C40:6+1.747×LysoPC 20:3+0.913×β-hydroxybutyric acid+0.939×Fumaric acid;
wherein the numeric value of each metabolite in the equation is the concentration in uM of the metabolites after median normalization, log transformation and auto-scaling.

28. The method of claim 27, wherein a stage I probability score that meets or exceeds a stage I threshold indicates that the subject has stage I non-small cell lung cancer.

29. The method of any one of claims 1 and 26 to 28, further comprising determining a stage II probability score for the biological sample according to the formula 3:
logit(P)=log(P/(1−P))=0.346+2.565×β-hydroxybutyric acid−2.219×citric acid+2.904×carnitine−1.599×PC ae C40:6;
wherein the numeric value of each metabolite in the equation is the concentration in uM of the metabolites after median normalization, log transformation and auto-scaling.

30. The method of claim 29, wherein a stage II probability score that meets or exceeds a stage II threshold indicates that the subject has stage II non-small cell lung cancer.

31. The method of any one of claims 1 and 26 to 30, further comprising determining a stage/II probability score for the biological sample according to the formula 5:
logit(P)=log(P/(1−P))=2.346−1.528×PC ae C40:6+1.429×β-hydroxybutyric acid−2.481×citric acid+1.03×LysoPC 20:3+1.773×fumaric acid
wherein the numeric value of each metabolite in the equation is the concentration in uM of the metabolites after median normalization, log transformation and auto-scaling.

32. The method of claim 31, wherein a stage I/II probability score that meets or exceeds a stage I/II threshold indicates that the subject has stage I or stage II non-small cell lung cancer.

33. The method of any one of claims 26 to 32, wherein the subject is a non-smoker.

34. The method of claim 26, wherein the subject is a smoker.

35. The method of claim 34, further comprising determining a stage I probability score for the biological sample according to the formula 2:
logit(P)=log(P/(1−P))=0.311+0.641×Amount of Smoking−1.372×PC ae C40:6+1.623×LysoPC 20:3+0.882×β-hydroxybutyric acid+0.65×fumaric acid
wherein the numeric value of each metabolite in the equation is the concentration in uM of the metabolites after median normalization, log transformation and auto-scaling.

36. The method of claim 35, wherein a stage I probability score that meets or exceeds a stage I threshold indicates that the subject has stage I non-small cell lung cancer.

37. The method of claim 34, 35, or 36, further comprising determining a stage II probability score for the biological sample according to the formula 4:
logit(P)=log(P/(1−P))=0.098+1.489×Amount of smoking+2.911×β-hydroxybutyric acid−1.627×Citric acid+2.605×Carnitine−0.702×PC ae C40:6;
wherein the numeric value of each metabolite in the equation is the concentration in uM of the metabolites after median normalization, log transformation and auto-scaling.

38. The method of claim 37, wherein a stage II probability score that meets or exceeds a stage II threshold indicates that the subject has stage II non-small cell lung cancer.

39. The method of any one of claims 34 to 38, further comprising determining a stage I/II probability score for the biological sample according to the formula 6:
logit(P)=log(P/(1−P))=2.427+1.425×Amount of smoking−1.414×PC ae C40:6+1.414×βhydroxybutyric acid−2.193×citric acid+1.738×LysoPC 20:3+1.44×fumaric acid;
wherein the numeric value of each metabolite in the equation is the concentration in uM of the metabolites after median normalization, log transformation and auto-scaling.

40. The method of claim 39, wherein a stage I/II probability score that meets or exceeds a stage I/II threshold indicates that the subject has stage I or stage II non-small cell lung cancer.

41. The method of any one of claims 1 to 40, wherein the method is a method of diagnosing non-small cell lung cancer

42. The method of claim 42, wherein the non-small cell lung cancer is stage I or stage II non-small cell lung cancer.

43. A method, the method comprising determining the concentration of each metabolite of a group of metabolites in a biological sample from a subject, wherein the group of metabolites comprises β-hydroxybutyric acid, LysoPC 20:3, fumaric acid, and spermine.

44. The method of claim 43, wherein the group of metabolites consists of β-hydroxybutyric acid, LysoPC 20:3, fumaric acid, and spermine.

45. The method of claim 43 or 44, further comprising determining a probability score for the biological sample according to the formula 7:
logit(P)=log(P/(1−P))=0.504+2.192×LysoPC 20:3+2.252×β-hydroxybutyric acid+1.23×fumaric acid−1.798×spermine;
wherein the numeric value of each metabolite in the equation is the concentration in uM of the metabolites after median normalization, log transformation and auto-scaling.

46. The method of claim 45, wherein a probability score that meets or exceeds a stage I threshold indicates that the subject has stage I non-small cell lung cancer.

47. The method of any one of claims 43 to 46, wherein the subject is a non-smoker.

48. The method of claim 43 or 44, wherein the subject is a smoker.

49. The method of claim 48, further comprising determining a probability score for the biological sample according to the formula 8:
0.739+0.68×fumaric acid−1.861×spermine+5.248×period of smoking−4.19×Cig/day+1.139×β-hydroxybutyric acid+1.776×LYSO-PC 20:3;
wherein the numeric value of each metabolite in the equation is the concentration in uM of the metabolites after median normalization, log transformation and auto-scaling.

50. The method of claim 49, wherein a probability score that meets or exceeds a stage I threshold indicates that the subject has stage I non-small cell lung cancer.

51. The method of any one of claims 5, 9, 13, 17, 21, 25, 28, 30, 32, 36, 38, 40, 42, 46, and 50, further comprising treating the subject for lung cancer.

52. The method of claim 51, wherein treating the subject for lung cancer comprises administering a therapeutic agent to the subject.

53. The method of claim 52, wherein the therapeutic agent comprises: Cisplatin; Carboplatin; Paclitaxel; Albumin-bound paclitaxel; Docetaxel; Gemcitabine; Vinorelbine; Etoposide; Pemetrexed; Bevacizumab; Ramucirumab; Erlotinib; Afatinib; Gefitinib; Osimertinib; Dacomitinib; Necitumumab; Crizotinib; Ceritinib; Lorlatinib; Entrectinib; Dabrafenib; Trametinib; Selpercatinib; pralsetinib; Capmatinib; Larotrectinib; entrectinib; Nivolumab; pembrolizumab; atezolizumab; Durvalumab; Ipilimumab; or combinations thereof.

54. Use of a therapeutic agent to treat a subject diagnosed with non-small cell lung cancer according to a method as defined in any one of claims 5, 9, 13, 17, 21, 25, 28, 30, 32, 36, 38, 40, 42, 46, and 50 wherein the therapeutic agent is Cisplatin; Carboplatin; Paclitaxel; Albumin-bound paclitaxel; Docetaxel; Gemcitabine; Vinorelbine; Etoposide; Pemetrexed; Bevacizumab; Ramucirumab; Erlotinib; Afatinib; Gefitinib; Osimertinib; Dacomitinib; Necitumumab; Crizotinib; Ceritinib; Lorlatinib; Entrectinib; Dabrafenib; Trametinib; Selpercatinib; pralsetinib; Capmatinib; Larotrectinib; entrectinib; Nivolumab; pembrolizumab; atezolizumab; Durvalumab; Ipilimumab; or combinations thereof.

55. A therapeutic agent for treating a subject diagnosed with non-small cell lung cancer according to a method as defined in any one of claims 5, 9, 13, 17, 21, 25, 28, 30, 32, 36, 38, 40, 42, 46, and 50, wherein the therapeutic agent is Cisplatin; Carboplatin; Paclitaxel; Albumin-bound paclitaxel; Docetaxel; Gemcitabine; Vinorelbine; Etoposide; Pemetrexed; Bevacizumab; Ramucirumab; Erlotinib; Afatinib; Gefitinib; Osimertinib; Dacomitinib; Necitumumab; Crizotinib; Ceritinib; Lorlatinib; Entrectinib; Dabrafenib; Trametinib; Selpercatinib; pralsetinib; Capmatinib; Larotrectinib; entrectinib; Nivolumab; pembrolizumab; atezolizumab; Durvalumab; Ipilimumab; or combinations thereof.

56. The method of any one of claims 1 to 53, wherein the sample is plasma.

57. The method of any one of claims 1 to 53, wherein the sample is serum.

58. The method of any one of claims 1 to 53, wherein the sample is blood or a blood product.
